(19) United States
(12) Patent Application Publication
(10) Pub. No.: US 2018/0305656 A1
(43) Pub. Date: Oct. 25, 2018

(54) METHODS OF PRESERVING A MICROALGAE BIOMASS AND A PRESERVED MICROALGAE BIOMASS

(71) Applicant: BATTELLE ENERGY ALLIANCE, LLC, Idaho Falls, ID (US)

(72) Inventors: LYNN M. WENDT, IDAHO FALLS, ID (US); BRADLEY D. WAHLEN, RIGBY, ID (US); CHENLIN LI, IDAHO FALLS, ID (US)

(21) Appl. No.: 15/495,625

(22) Filed: Apr. 24, 2017

(51) Int. Cl. C12N 1/12; C12P 7/46; C12N 1/20

(52) U.S. Cl. CPC C12N 1/12; C12N 1/20; C12P 7/46

(57) ABSTRACT

A method of preserving a biomass. The method comprises adding an acid solution to a biomass comprising microalgae to form an acidified microalgae biomass composition. The acidified microalgae biomass composition is stored under anaerobic conditions without inoculating the acidified microalgae biomass composition with bacteria. An additional method of preserving a biomass comprises storing the acidified microalgae biomass composition under anaerobic conditions and exposing the acidified microalgae biomass composition to carbon dioxide, nitrogen, or a combination thereof to produce a coproduct comprising succinic acid. Yet another method of preserving a biomass comprises storing the acidified microalgae biomass composition under anaerobic conditions and in the presence of carbon dioxide. A preserved biomass is also disclosed.

### STATEMENT REGARDING FEDERALLY SPONSORED RESEARCH OR DEVELOPMENT

This invention was made with government support under Contract Number DE-AC07-05ID14517 awarded by the United States Department of Energy. The government has certain rights in the invention.

### TECHNICAL FIELD

The disclosure, in various embodiments, relates generally to preservation of microalgae. More specifically, the disclosure, in various embodiments, relates to methods of preserving a microalgae biomass and the preserved microalgae biomass.

### BACKGROUND

Algal biomass is becoming increasingly attractive as a feedstock for biofuel production and other uses. Algae have a faster growth rate and productivity than conventional terrestrial crops. The grown algae provide high-energy area yields and require less land to grow than conventional terrestrial crops. The algae can also be cultivated in non-arable areas, such as in fresh water, brackish water, salt water, or waste water. As with many other types of biomass, there is a seasonality to the production of the algal biomass. In many geographical areas, the production of the algal biomass may occur year-round. However, the growth rate and yield fluctuate due to changes in temperature and solar irradiation. The fluctuation in production between summer and winter months poses a challenge for delivering a predictable, constant feedstock supply to a conversion facility. During the summer months, when algal biomass productivity is highest, production may exceed conversion capacity, resulting in delayed processing. However, the algal biomass is susceptible to degradation following harvesting due to oxygen (O₂) and moisture exposure. Therefore, the algal biomass is ideally used immediately after harvesting. The seasonal variability and the risk of algal biomass degradation increases uncertainty in algal biomass productivity and increases risks to feedstock supply for conversion.

Drying has been used to stabilize the algal biomass for long term storage. Any algal biomass produced in excess during the high productivity summer months is dried and stored for utilization during low productivity months. However, drying is energy intensive, costly, and produces greenhouse gases.

Wet, anaerobic storage, e.g., ensiling, has been utilized to preserve many types of herbaceous biomass including corn stover, wheat straw, sweet sorghum, switchgrass, and other grasses. Ensiling is a widely used method of preserving herbaceous crops, where lactic fermentation of soluble sugars by Lactobacillus sp. produces organic acids (e.g., lactic acid, acetic acid), lowering the pH of the stored herbaceous biomass. The reduced pH limits further microbial degradation and stabilizes the herbaceous biomass. Ensiling has also been used to preserve macroalgae. Small quantities of microalgae (less than 2.5%) have also been added to silage in order to increase the protein content for feed or for increased yield in anaerobic digestion.

### BRIEF SUMMARY

An embodiment of the disclosure comprises a method of preserving a biomass. The method comprises adding an acid solution to a biomass comprising microalgae to form an acidified microalgae composition. The acidified microalgae biomass composition is stored under anaerobic conditions without inoculating the acidified microalgae composition with a bacteria.

Another embodiment of the disclosure comprises a method of preserving a biomass. The method comprises adding an acid solution to a biomass comprising microalgae to form an acidified microalgae biomass composition. The acidified microalgae biomass composition is stored under anaerobic conditions and then exposed to carbon dioxide, nitrogen, or a combination thereof to produce a coproduct comprising succinic acid.

Still another embodiment of the disclosure comprises a method of preserving a biomass. The method comprises adding an acid solution to a biomass comprising microalgae to form an acidified microalgae biomass composition. The acidified microalgae biomass composition is stored under anaerobic conditions and in the presence of carbon dioxide.

Yet another embodiment of the disclosure comprises a preserved biomass that comprises a biomass comprising microalgae and succinic acid, the succinic acid comprising up to about 14% of the biomass on a dry weight basis.

### BRIEF DESCRIPTION OF THE DRAWINGS

FIGS. 1-4 are photographs of samples of microalgae biomass and microalgae biomass blends treated with various acids according to embodiments of the disclosure;

FIG. 5 includes bar graphs showing the production of organic acids from microalgae biomass, microalgae biomass blends, and herbaceous biomass treated with various acids according to embodiments of the disclosure; and

FIG. 6 is a bar graph showing the production of succinic acid from acid-treated microalgae biomass samples and purged with nitrogen (N₂) or carbon dioxide (CO₂) according to embodiments of the disclosure.

### DETAILED DESCRIPTION

Methods of preserving e.g., stabilizing, a biomass, such as a microalgae biomass, are disclosed. The microalgae biomass is preserved using a wet storage process, which lowers the cost of preserving the microalgae biomass compared to a drying process. No drying of the microalgae biomass is utilized for the wet storage process. The wet storage process stabilizes the metabolic activity of the microalgae biomass, enabling use of the microalgae biomass months after its harvest. By preserving the microalgae biomass, the microalgae biomass may be stored during low productivity months, such as during winter months, and subsequently used as a feedstock. Thus, the preserved microalgae biomass may be used to meet seasonal demands for the microalgae biomass and a biorefinery that utilizes the microalgae biomass as a feedstock may be operated at about 100% capacity or close to near capacity year round. The wet storage process of the microalgae biomass may ensile the microalgae and also produces one or more commercially valuable coproducts, such as succinic acid ((CH₂)₂(CO₂H)₂) or other organic acid. The coproducts upgrade the microalgae biomass in addition to preserving the microalgae biomass. The microalgae biomass may be preserved and the coproducts produced concurrently utilizing the wet storage process. A herbaceous biomass may, optionally, be used in the wet storage process to improve the preservation of the microalgae biomass. A preserved biomass is also disclosed.

As used herein, the term “microalgae” means and includes unicellular, eukaryotic organisms or cyanobacteria that are capable of photoautotrophic growth where solar energy is used to fix carbon dioxide (CO₂) into organic compounds (e.g., sugars) with the concomitant release of oxygen (O₂). The microalgae may be a naturally occurring species, a genetically selected strain, a genetically manipulated strain, a transgenic strain, a synthetic microalgae, or combinations thereof. The microalgae may include, but is not limited to, green algae, brown algae, red algae, or combinations thereof. The microalgae may include a single species or strain of microalgae, or a combination of species or strains, such as those grown in a polyculture. By way of example only, the microalgae may include, but is not limited to, Chlorella, Spirulina, Phaeophyta, Coelastrum, Micractinium, Nannochloropsis, Porphyridium, Nostoc, Haematococcus, Chlorophyta, Rhodophyta, Dunaliella, Scenedesmus, Microcystis, Synechocystis, Anabaena, Chlamydomonas, Oedogonium, or combinations thereof. In some embodiments, the microalgae are Scenedesmus, such as Scenedesmus obliquus or Scenedesmus acutus.

As used herein, the term “biomass” means and includes a biological material that can be converted into a biofuel, chemical, or other product.

As used herein, the term “herbaceous” means and includes a cellulosic biomass that contains sugar polymers and lignin and may be at low or high moisture content. The herbaceous biomass may be derived from an agricultural crop, crop residue, trees, woodchips, sawdust, paper, cardboard, grasses, yard waste, or combinations thereof. The herbaceous biomass may include, but is not limited to, corn stover, grass clippings, grain sorghum residue, biomass sorghum, or combinations thereof.

As used herein, the term “preserved” or “stabilized” means and includes maintaining the metabolic activity of the microalgae biomass or microalgae biomass blend without substantially degrading the microalgae biomass or microalgae biomass blend. For instance, the metabolic activity of the microalgae biomass may be stabilized for at least one week, such as at least two weeks, while exhibiting a dry matter loss of less than about 30% dry basis, such as less than about 25% dry basis, less than about 20% dry basis, less than about 15% dry basis, less than about 10% dry basis, less than about 5% dry basis, or less than about 2% dry basis. By way of example only, the metabolic activity of the microalgae biomass may be stabilized for one month or more, two months or more, three months or more, four months or more, five months or more, or six months or more, while exhibiting a dry matter loss of less than about 15% dry basis. In some embodiments, the loss in dry matter may be less than about 10% dry basis, less than about 5% dry basis, or less than about 2% dry basis.

As used herein, the term “anaerobic” means and includes an amount of oxygen in an environment that is less than about 10% of saturation for dissolved oxygen. By way of example only, the amount of oxygen may be less than about 5% of saturation for dissolved oxygen, less than about 4% of saturation for dissolved oxygen, less than about 3% of saturation for dissolved oxygen, less than about 2% of saturation for dissolved oxygen, or less than about 1% of saturation for dissolved oxygen.

The preserved microalgae biomass may improve the process of producing and converting the microalgae biomass to a biofuel. The preserved microalgae biomass may be used as a feedstock for a bioprocess, e.g., a fuel conversion technology, such as hydrothermal liquefaction for microalgae or microalgae/herbaceous biomass blends conversion, algal lipid extraction and upgrading, and carbohydrate/protein microbial fermentation followed by hydrothermal liquefaction. The conversion of the preserved microalgae biomass into the biofuel may be conducted in the biorefinery by conventional techniques, which are not described in detail herein. By preserving the microalgae biomass, the feedstock supply chain may be consistent and the risk of running the biorefinery at less than full capacity is reduced. The preserved microalgae biomass may be easily transported from its harvest site and stored until use at an on-site storage facility located in proximity to the biorefinery or used at a centrally located biorefinery. By including a low cost herbaceous biomass, such as yard waste, with the microalgae biomass, a low cost feedstock for the bioprocess may be produced. The preserved microalgae biomass may also be used as fodder or feed for livestock. By tailoring the ratio of microalgae biomass to herbaceous biomass, the protein content of the fodder or feed may be optimized.

Since the process is a wet process, less water removal is needed, decreasing the cost of the process, the energy used in the process, and greenhouse gas emissions. The wet storage process of the disclosure may preserve the metabolic activity of the microalgae biomass without substantial dry matter loss. The wet storage process of the disclosure may, thus, extend the storage period for the microalgae biomass, such as up to six or more months compared to days without preserving the microalgae biomass. In some embodiments, the wet storage process of the disclosure preserves the metabolic activity of the microalgae biomass for at least about 30 days (e.g., about one month), with a dry matter loss of less than about 15% dry basis (db) during the storage period. The wet storage process of the disclosure may enable shorter term preservation of the microalgae biomass during formation of the coproduct(s), such as for about one week or about two weeks.

The microalgae biomass may be produced and harvested by conventional techniques, which are not described in detail herein. After harvesting, the microalgae biomass may be chopped or comminuted into smaller pieces. The harvested microalgae biomass may have a neutral pH, such as a pH of about 7.0 or greater, a pH of about 8.0 or greater, or a pH of from about 7.0 to about 8.0. The microalgae biomass may, optionally, be concentrated, e.g., water removed, before use in the wet storage process. The concentrated microalgae biomass includes solids and may have a moisture content of from about 50% wet basis (wb) to about 90% (wb), such as a moisture content of from about 60% wet basis to about 80% wet basis. The moisture content is determined by drying to a constant weight at 105° C. In some embodiments, the concentrated microalgae biomass has a paste-like consistency and a solids content of about 20%, which is conventional for microalgae biomass that is to be converted into a fuel, such as a biofuel. However, the concentrated microalgae biomass may have a higher solids content or a lower solids content. In some embodiments, the moisture content of the concentrated microalgae biomass is about 80% wet basis.

Before preserving the microalgae biomass, at least one herbaceous biomass may, optionally, be combined with the microalgae biomass. The herbaceous biomass may be produced and harvested by conventional techniques, which are not described in detail herein. For convenience and simplicity, the term “microalgae biomass blend” is used herein to refer to a biomass having the microalgae biomass and the at least one herbaceous biomass. The microalgae biomass or the microalgae biomass blend may be preserved using the wet storage process according to embodiments of the disclosure. While specific embodiments herein describe a blend of microalgae and corn stover, another herbaceous biomass may be used in place of the corn stover. The ratio of the microalgae biomass to the herbaceous biomass in the microalgae biomass blend may range from about 5% (dry basis (db)) to about 40% (db) of the microalgae biomass:from about 60% (db) to about 95% (db) of the herbaceous biomass. The ratio of the microalgae biomass to the herbaceous biomass may be tailored depending on the desired use of the preserved microalgae biomass blend. In some embodiments, the ratio of the microalgae biomass to the herbaceous biomass in the microalgae biomass blend is about 20% (db):about 80% (db). The herbaceous biomass may be combined with the microalgae biomass, such as by simple mixing, before preserving the microalgae biomass blend. By way of example only, if the herbaceous biomass is a dry solid, the herbaceous biomass may be rehydrated with water prior to combining the herbaceous biomass and microalgae biomass. The microalgae biomass blend may have a moisture content of from about 50% wet basis to about 90% wet basis, such as a moisture content of from about 60% wet basis to about 80% wet basis. The microalgae biomass and the herbaceous biomass may be combined in a vessel in which the wet storage process is to be conducted.

The microalgae biomass or microalgae biomass blend may be preserved from immediately after harvesting up until about 72 hours after harvesting. To preserve the microalgae biomass or microalgae biomass blend, the microalgae biomass or microalgae biomass blend may be acidified, such as by adding an acid, acid salt, or an acid source compound to the microalgae biomass or microalgae biomass blend. The acid may be an organic acid or an inorganic acid, the acid salt may be an organic acid salt or an inorganic acid salt, such as sodium bisulfate or sodium sulfate, and the acid source compound may be an organic acid source compound or an inorganic acid source compound. The acid may be lactic acid, acetic acid, sulfuric acid, formic acid, propionic acid, citric acid, benzoic acid, hexanoic acid, succinic acid, or combinations thereof. The acid source compound may be a chemical compound that reacts with another compound in the microalgae biomass or microalgae biomass blend to produce one of the above-mentioned acids. The microalgae biomass or microalgae biomass blend may be contacted with the acid or acid source compound, such as by adding an acid solution to a vessel containing the microalgae biomass or microalgae biomass blend. The acid solution may be an aqueous solution of the acid, of the acid salt, or of the acid source compound. The pH of the acid solution may range from about 1.0 to about 5.0, such as from about 1.0 to about 4.0, from about 1.0 to about 3.0, from about 2.0 to about 4.0, from about 3.0 to about 5.0, or from about 1.0 to about 2.0. In some embodiments, the pH of the acid solution is about 2.0. In other embodiments, the pH of the acid solution is about 4.0.

A sufficient volume of the acid solution may be added to the microalgae biomass or the microalgae biomass blend to produce an acidified microalgae biomass composition having the desired pH and a moisture content of from about 50% wet basis to about 90% wet basis. It is understood that the acidified microalgae biomass composition may include the microalgae biomass or the microalgae biomass blend. The addition of the acid solution may reduce the pH of the acidified microalgae biomass composition to less than about 7.0. The acid solution may be added to the microalgae biomass or the microalgae biomass blend such that the pH of the acidified microalgae biomass composition is less than about 7.0, such as less than about 6.0, less than about 5.0, less than about 4.0, less than about 3.0, less than about 2.0, or less than about 1.0. The pH of the acidified microalgae biomass composition may be from about 1.0 to about 6.0, from about 1.0 to about 5.0, from about 1.0 to about 4.0, from about 1.0 to about 3.0, from about 2.0 to about 4.0, from about 2.0 to about 5.0, from about 3.0 to about 5.0, from about 1.0 to about 2.0, from about 1.5 to about 4.5, from about 1.5 to about 3.5, from about 1.5 to about 2.5, from about 2.0 to about 4.0, from about 2.5 to about 4.5, from about 3.0 to about 4.0, or from about 4.0 to about 5.0. Upon reaching the desired pH, growth of microorganisms, such as bacteria, mold, and yeast, in the microalgae biomass or microalgae biomass blend may be inhibited and the microalgae biomass or microalgae biomass blend is preserved. In some embodiments, the pH of the acidified microalgae biomass composition is between about 2.0 and about 3.0. In other embodiments, the pH of the acidified microalgae biomass composition is between about 3.0 and about 4.0. In yet other embodiments, the pH of the acidified microalgae biomass composition is between about 4.0 and about 5.0. The pH of the acidified microalgae biomass composition may be maintained in this range for about one month or more. In some embodiments, the acid solution is added to the microalgae biomass or the microalgae biomass blend to achieve a moisture content of about 60% wet basis. The vessel in which the microalgae biomass or microalgae biomass blend is acidified may be a conventional reactor, which is not described in detail herein.

After acidification, the acidified microalgae biomass composition may be stored and maintained in the vessel under anaerobic conditions. By way of example only, the vessel containing the acidified microalgae biomass composition may be sealed and purged to remove air, preventing exposure of the acidified microalgae biomass composition to oxygen (O₂). The vessel may also contain sufficient biomass such that limited airspace is remaining in the vessel. A sufficient volume of the acidified microalgae biomass composition may be added to the vessel with mechanical compaction to achieve the limited airspace in the vessel. The acidified microalgae biomass composition may be purged with carbon dioxide, nitrogen, or combinations thereof. In some embodiments, carbon dioxide is used to purge the acidified microalgae biomass composition. The vessel may be stored at room temperature (from about 20° C. to about 25° C.) and in the dark for the desired storage period. The vessel in which the acidified microalgae biomass composition is stored and maintained under anaerobic conditions may be the same as or different from the vessel in which the microalgae biomass or microalgae biomass blend is acidified.

The carbon dioxide, nitrogen, or combinations thereof introduced into the vessel may produce the coproduct during storage of the acidified microalgae biomass composition, which includes the microalgae biomass or microalgae biomass blend. The coproduct may be a commercially valuable coproduct that increases the value of the preserved microalgae biomass or microalgae biomass blend. The coproduct may be a specialty chemical compound, such as a precursor used in a pharmaceutical, nutraceutical, or other industry. These coproducts can be precursors to other fuel products and offer a valuable coproduct stream for biorefineries. The coproduct may be a solid, liquid, or a gas. The solid or liquid coproduct may include, but is not limited to, succinic acid, butanediol, lactic acid, acetic acid, propionic acid, butyric acid, glycerol, ethanol, or combinations thereof. The gaseous coproduct may include, but is not limited to, hydrogen, methane, or a combination thereof. By way of example only, when the microalgae biomass is stored alone, the anaerobic metabolism may produce succinic acid, butanediol, lactic acid, acetic acid, propionic acid, butyric acid, glycerol, ethanol, or combinations thereof.

The introduction of the carbon dioxide, nitrogen, or a combination thereof into the vessel containing the acidified microalgae biomass composition may increase the amount of succinic acid or other coproduct produced during the wet storage process compared to conducting the wet storage process without the introduction of the carbon dioxide, nitrogen, or a combination thereof. In addition to succinic acid, acetic acid, propionic acid, lactic acid, butyric acid, or a combination thereof may be produced. Thus, the preserved microalgae biomass or preserved microalgae biomass blend may include higher amounts of the succinic acid or other coproducts than were present in the microalgae biomass or microalgae biomass blend before conducting the wet storage process. The production of succinic acid or other coproducts was unexpected and surprising since no succinic acid fermentative bacteria (e.g., Actinobacillus succinogenes) inoculation was conducted during the wet storage process. In some embodiments, the succinic acid is present in the preserved microalgae biomass or preserved microalgae biomass blend at up to 14% on a dry weight basis.

The coproduct, such as succinic acid, may also be produced when the acidified microalgae biomass composition is stored and maintained under the anaerobic conditions where the vessel has a headspace above the acidified microalgae biomass composition. The headspace may provide a sufficient volume for the microalgae biomass to expand in volume during the wet storage process. The headspace may be produced by introducing a desired volume of the acidified microalgae biomass composition into the vessel to partially fill the vessel and then evacuating the vessel. Alternatively, the headspace may be produced by partially filling the vessel with the desired volume of the acidified microalgae biomass composition and then introducing CO₂ or nitrogen into the vessel. Without being bound to any theory, it is believed that as the microalgae biomass of the acidified microalgae biomass composition degrades, CO₂ is produced, which CO₂ is subsequently consumed by the microalgae to produce the coproduct, such as the succinic acid. To produce the coproduct, the acidified microalgae biomass composition may be stored in the vessel having headspace under the anaerobic conditions and in an environment comprising CO₂, consisting essentially of CO₂, or consisting of CO₂. This environment in the vessel may be maintained by preventing air infiltration into the vessel.

The acidified microalgae biomass composition may be stored under the carbon dioxide, nitrogen, or a combination thereof for an amount of time sufficient to produce the succinic acid or other coproduct without experiencing significant dry matter loss of the microalgae biomass or the microalgae biomass blend or a decrease in the metabolic activity of the microalgae biomass or the microalgae biomass blend. In some embodiments, the acidified microalgae biomass composition is purged with carbon dioxide. The wet storage process of the disclosure may preserve the metabolic activity of the microalgae biomass without substantial dry matter loss, such as less than about 15% dry basis over the storage period. In some embodiments, the dry matter loss is less than about 10% dry basis over the storage period, less than about 5% dry basis over the storage period, or less than about 2% dry basis over the storage period. The carbon dioxide, nitrogen, or a combination thereof may be maintained in the vessel until a maximum amount of the coproduct is produced without experiencing a significant loss of the dry matter or a decrease in the metabolic activity of the microalgae biomass or of the microalgae biomass blend. In some embodiments, the wet storage process of the disclosure enables the microalgae biomass or the microalgae biomass blend to be stored for up to about six months or more. The wet storage process of the disclosure preserves the metabolic activity of the microalgae biomass or the microalgae biomass blend for at least about 30 days, with less than about 5% dry matter loss.

Without being bound to any theory, it is believed that the CO₂ enables the production of the succinic acid. Since the microalgae biomass remains metabolically active during the wet storage process, sugars, carbohydrates, proteins, lipids, or combinations thereof present in the acidified microalgae biomass composition may be converted to succinic acid. By way of example only, glucose in the acidified microalgae biomass composition may be converted to succinic acid by way of pyruvate carboxylase or phosphoenolpyruvate carboxykinase and the reductive branch of the citric acid cycle (i.e., the Krebs cycle or tricarboxylic acid (TCA) cycle). Thus, the succinic acid is produced in-situ by the microalgae biomass or microalgae biomass blend. It was surprising and unexpected that the succinic acid was produced in-situ since no succinic acid was used to acidify the microalgae biomass or the microalgae biomass blend. If, however, succinic acid is used to acidify the microalgae biomass or the microalgae biomass blend, an increased amount of succinic acid is present following the conversion relative to the amount of succinic acid used to acidify the microalgae biomass or the microalgae biomass blend. While lactic acid has been produced from macroalgae (i.e., seaweed) biomass, inoculating the macroalgae biomass with lactic acid bacteria (LAB) was required because the macroalgae are not metabolically active after harvesting. Therefore, although lactic acid is produced, the lactic acid is not produced in-situ by the macroalgae biomass. Rather, the macroalgae biomass requires the LAB inoculation to produce the lactic acid.

The coproduct may then be recovered from the acidified microalgae biomass composition including the microalgae biomass or microalgae biomass blend. If the coproduct is a solid or liquid, the coproduct may be recovered by conventional separation techniques including, but not limited to, filtration, crystallization, decantation, sublimation, evaporation, simple distillation, fractional distillation, extraction, or chromatography. If the coproduct is gaseous, the coproduct may be recovered by enabling the gaseous coproduct to be selectively removed from the vessel. The wet storage process according to embodiments of the disclosure may produce the coproduct at a yield of up to about 0.34 g of coproduct per gram of sugar, carbohydrate, protein, or lipid present in the microalgae biomass or microalgae biomass blend. By way of example only, succinic acid may be produced at up to about 14% on a dry weight basis (i.e., up to about 14% succinic acid per gram of microalgae biomass), such as from about 6% on a dry weight basis to about 14% on a dry weight basis. For instance, when the vessel includes a headspace during the wet storage process, up to about 10% by weight of the succinic acid may be produced. By recovering the commercially valuable coproduct, the coproduct may be sold to offset costs of the wet storage process, such as storage cost. In some embodiments, the coproduct is succinic acid, which is a precursor for multiple, high-value commodity chemicals.

Without being bound to any theory, it is believed that the limited oxygen during the wet storage process enables anaerobic fermentation of sugars, carbohydrates, proteins, or lipids present in the acidified microalgae biomass composition into the coproduct(s), such as the organic acid(s). The presence of the organic acid decreases the pH of the environment, limits microbial degradation, and stabilizes the microalgae biomass or the microalgae biomass blend. The produced organic acid(s) may include, but is not limited to, acetic acid, propionic acid, lactic acid, succinic acid, or butyric acid. The organic acid produced by the wet storage process may decrease the pH of the acidified microalgae biomass composition beyond the pH achieved by the addition of the acid solution to the microalgae biomass or the microalgae biomass blend. The organic acid maintains the acidic pH and serves to inhibit the growth of microorganisms responsible for excessive degradation of the microalgae biomass, including yeast, mold, and bacteria (e.g., Clostridium sp.)

Without being bound to any theory, it is believed that sugars, carbohydrates, proteins, or lipids present in the herbaceous biomass may increase anaerobic fermentation when used in combination with the microalgae biomass. Additionally, including the herbaceous biomass with the microalgae biomass is advantageous because rheological properties of the herbaceous biomass are maintained such that conventional apparatus and technologies for herbaceous biomass may be utilized with the microalgae biomass blend. By way of example only, conventional storage structures ranging from small-scale silage bags to bunkers and drive over piles may be used in the wet storage process according to embodiments of the disclosure.

The preserved microalgae biomass or preserved microalgae biomass blend may be stored for one month or more, such as two months or more, three months or more, four months or more, five months or more, or six months or more, without experiencing high levels of dry matter loss. The metabolic activity of the preserved microalgae biomass or preserved microalgae biomass blend may be maintained during the storage period, while low levels of dry matter loss are observed. The preserved microalgae biomass or preserved microalgae biomass blend may exhibit a dry matter loss of less than about 15% dry basis during storage, such as from about 6.5% dry basis to about 14.5% dry basis. In comparison, control microalgae biomass compositions that were not acidified but were stored under anaerobic conditions exhibited a dry matter loss of about 37% dry basis during storage. Control microalgae biomass compositions that were not acidified and were stored under aerobic conditions also exhibited a greater dry matter loss than the acidified microalgae biomass compositions. The microalgae biomass or microalgae biomass blend acidified with acetic acid and lactic acid exhibited a dry matter loss of from about 6.8% dry basis to about 8.8% dry basis, while the microalgae biomass or microalgae biomass blend acidified with sulfuric acid exhibited a dry matter loss of from about 12% dry basis to about 14% dry basis.

To further increase the preservation of the microalgae biomass or microalgae biomass blend, the acidified microalgae biomass composition may, optionally, be inoculated with bacteria, such as lactic acid bacteria (LAB), before storing the acidified microalgae biomass composition under the anaerobic conditions. However, the wet storage process according to embodiments of the disclosure may be conducted without an LAB, or other bacteria, inoculation.

The following examples serve to explain embodiments of the disclosure in more detail. These examples are not to be construed as being exhaustive or exclusive as to the scope of the disclosure.

### EXAMPLES

### Example 1

Scenedesmus Biomass

Scenedesmus obliquus was cultivated in 1,000 L outdoor raceway ponds at the Regional Algal Feedstock Testbed located at the University of Arizona in Tucson, Ariz. The media contained 0.134 g/L NaNO₃; 0.075 g/L MgSO₄ (7H₂O); 0.013 g/L KH₂PO₄; 0.175 g/L potash; 0.0054 g/L Fecitraplex; 0.0029 g/L H₃BO₃; 0.0018 g/L MnCl₂ (4H₂O); 0.0014 g/L ZnSO₄ (7H₂O): 0.0004 g/L Na₂MoO₄ (2H₂O); 0.00008 g/L CuSO₄ (5H₂O); 0.00006 g/L Co(NO₃)₂ (6H₂O); and 0.0001 g/L NiCl₂ (6H₂O). The pH of the feedstock was maintained at 8.0 with CO₂ injection. The microalgae biomass was concentrated by centrifugation (Evodos 10, Raamsdonksveer, The Netherlands) to a paste with a moisture content of 80% wet basis (wb), as determined by drying to a constant weight at 105° C. The algae biomass was transported overnight to the Idaho National Laboratory in a cooler on ice. The microalgae biomass was immediately used for experiments upon arrival.

### Example 2

Corn Stover Biomass

Single pass corn stover was collected in Boone County, Iowa and was ground to pass through a 1-inch sieve using a Vermeer BG480 grinder (Pella, Iowa) and a Bliss Hammermill (Ponca City, Okla.) with no screen. In preparation for storage experiments, the corn stover was further size reduced to pass through a 6-mm screen with a Wiley Mill (model 4, Thomas, Swedesboro, N.J.).

### Example 3

Microalgae/Corn Stover Biomass Blend

Prior to blending, the dried corn stover was rehydrated for 24 hours with sufficient water to result in a 20:80 (dry basis, db) algae to corn stover blend with 60% moisture. At blending, sucrose was added to a concentration of 2% (db) of the total biomass to simulate soluble sugars that would be present in freshly chopped corn stover.

### Example 4

Storage, Dry Matter Loss, and pH Experiments

The effect of acid was examined by treating the microalgae biomass and microalgae biomass blend described above with various acids. Storage experiments were conducted with microalgae/corn stover blends and with microalgae unblended as a control. Initial proof-of-principle experiments were conducted in 50 mL serum vials, where the microalgae biomass was blended with the corn stover biomass at a ratio of 20:80 (dry basis, db) microalgae to corn stover. The microalgae/corn stover blends were acidified with one of three acids or with a combination of acids (sulfuric acid, pH 4, sulfuric acid pH 2, 3% lactic acid, 5% lactic acid, a mixture of 1% acetic acid and 3% lactic acid, a mixture of 2% acetic acid and 6% lactic acid) to reach a moisture content of 60% (wb) and desired pH or acid concentration (% of dry biomass). The biomass samples were treated to simulate storage conditions typical of homolactic fermentation and heterolactic fermentation. Homolactic fermentation occurs when the primary fermentation product is lactic acid, whereas heterolactic fermentation involves the production of acetic acid and carbon dioxide in addition to lactic acid. The sulfuric acid was used to evaluate storage performance at low pH in the absence of added organic acids.

Once blended and acidified, 5 g of the microalgae biomass blends and 2 g of microalgae biomass alone were immediately transferred to 50 ml serum vials, capped with butyl rubber stoppers and aluminum seals, purged with high purity nitrogen gas for 10 minutes, and stored at room temperature for 30 days. Anaerobic controls for the microalgae biomass blends and the microalgae biomass alone were prepared in like manner without acidification. Aerobic controls for both the microalgae biomass blends and the microalgae biomass alone were placed in serum vials open to the atmosphere without acidification. Each experimental condition was performed in triplicate and each vial was stored in a dark environment to prevent continued photosynthesis. The storage experiments were conducted for 30 days in sealed vials purged at the beginning of the experiment with nitrogen gas.

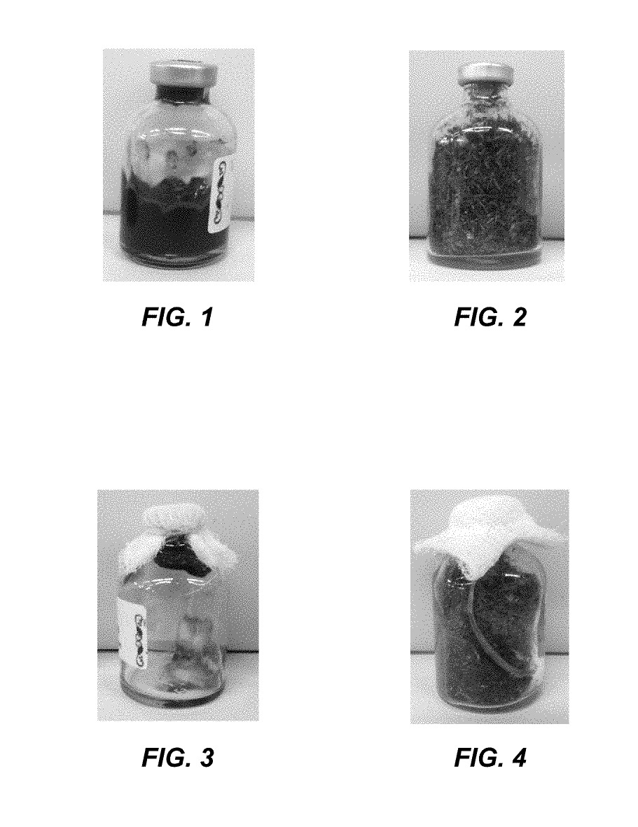

FIGS. 1-4 show the morphologies of the biomass samples after 30 days storage. Under the anaerobic, acidified conditions, the microalgae biomass alone (FIG. 1) and the microalgae biomass blend (FIG. 2) were observed with good stability and material preservation. In comparison, the non-acidified aerobic controls exhibited heavy mold (microalgae biomass only, FIG. 3) or filamentous growth (microalgae biomass blend, FIG. 4), indicating significant deterioration of the microalgae biomass or the microalgae biomass blend.

Experiments to evaluate natural ensiling were conducted in 125 mL jars (Thermo Scientific, Waltham, Mass.) outfitted with an S-type fermentation airlock that enabled fermentation gases to escape while isolating the contents of the jar from atmospheric oxygen. The fermentation airlock was affixed to the jar lid through the use of a silicon grommet (9.5 mm ID, 14.3 mm OD). Fermentation gases were collected in TEDLAR® bags (P/N # GSTP000-0606, Jensen Inert Products, Coral Springs, Fla.) connected to the outlet of the fermentation airlock for quantitation and compositional analysis.

To encourage ensiling, an inoculum of Lactobacillus acidophilus was added at a loading of 100,000 cfu per g of wet biomass. Once blended, the biomass was compacted into pre-weighed jars up to the top to limit headspace; approximately 32 g dry material equivalent was added to each jar. The jars with biomass were weighed and immediately sealed with the lid/fermentation airlock assembly. Water was added to the fermentation airlock and the TEDLAR® bag was attached to complete the seal. The storage reactors were stored at room temperature in the dark. The experiment was performed in quadruplicate. Moisture content of fresh and stored materials was determined gravimetrically after drying at 105° C. until reaching a constant weight. Dry matter loss for each experiment was determined based on the dry matter entering and leaving the storage reactor. Data from the LAB inoculated samples is indicated in the figures by the term “natural ensiling.”

The dry matter loss, initial pH and finial pH for the biomass samples are shown in Table 1. The microalgae biomass samples, both anaerobic and aerobic controls, where pH was not adjusted, exhibited high dry matter loss (43.9% and 36.8%, db, respectively). All acidified microalgae samples, in contrast, were better preserved, experiencing dry matter losses of 6.8% (db) to 14.2% (db), at least a 67% reduction compared to the controls. It should be noted that the 36.8% dry matter loss of the aerobic stored microalgae biomass included the dry mass of mold that had grown on the samples and may not reflect the real loss of the initial material, which could be much higher. The microalgae biomass samples treated with a mixture of acetic and lactic acids experienced the lowest dry matter loss (6.8% db to 8.8%, db), which was much lower than dry matter losses observed for the lactic acid treated microalgae biomass samples or the microalgae biomass samples treated with sulfuric acid (12% db-14% db), suggesting that the combination of lactic and acetic acid act to preserve the microalgae biomass in ways beyond a simple reduction in pH. The inhibitory effects imparted by mixtures of lactic and acetic acids may have added benefits to stability should the microalgae biomass or microalgae biomass blend become exposed to oxygen and when the microalgae biomass or microalgae biomass blend is reclaimed for conversion. The benefits of heterolactic fermentation come at a cost of higher dry matter loss compared to homolactic fermentation, as 1 mole of carbon dioxide is produced for each mole of lactic and acetic acid. In addition, a combination of lactic and acetic acid may have a negative impact on biochemical conversion of the stored biomass to fuel by inhibiting the microorganism responsible for conversion. This potential limitation may be overcome by carefully selecting a silage inoculant that is compatible with the intended conversion technology.

TABLE 1
Dry matter losses and pH changes of biomass samples stored anaerobically for 30 days.
Samples included corn stover, fresh microalgae, and microalgae/corn stover blends treated
with various acids to simulate conditions of ensiling or controls of the same
material stored directly without treatment.

| | | | | | | | | | | | Microalgae/ | |
|---|---|---|---|---|---|---|---|---|---|---|---|---|
| | | | | | | | | | | | Corn | |
| | Dry matter loss (%)^{a} | | | | | | Corn | | | | Stover | |
| | | | | | Algae/ | | Stover | | Microalgae | | blend | |
| Storage | Corn | | | | Corn | | Initial | Final | Initial | Final | Initial | Final |
| Conditions | stover | | Algae | | stover | | pH | pH | pH | pH | pH | pH |
| 3% lactic acid | 3.9 | (5.4)^{b} | 12.4 | (4.0) | 1.7 | (0.79) | 4.02 | 4.11 | 3.97 | 4.25 | 4.52 | 4.44 |
| 5% lactic acid | 4.4 | (0.04)^{b} | 14.2 | (4.8)^{b} | negligible | | 3.78 | 3.79 | 3.70 | 4.36 | 4.14 | 4.09 |
| 1% acetic, | negligible | | 8.8 | (0.80)^{b} | 0.6 | (0.22) | 4.03 | 4.61 | 3.69 | 4.38 | 4.19 | 4.40 |
| 3% lactic acid | | | | | | | | | | | | |
| 2% acetic, | 3.7 | (0.67) | 6.8 | (0.93) | negligible | | 3.76 | 4.21 | 3.38 | 4.30 | 3.66 | 3.80 |
| 6% lactic acid | | | | | | | | | | | | |
| Sulfuric acid, | 1.0 | (0.68) | 13.9 | (1.3)^{c} | 1.8 | (0.78) | 4.10 | 4.81 | 3.86 | 4.52 | 3.96 | 4.31 |
| pH~4 | | | | | | | | | | | | |
| Sulfuric acid, | 3.6 | (0.79) | 11.9 | (6.4)^{c} | 0.9 | (0.73)^{b} | 2.32 | 2.76 | 2.34 | 4.08 | 2.46 | 2.93 |
| pH~2 | | | | | | | | | | | | |
| Anaerobic | 3.4 | (2.8) | 43.9 | (0.79) | 7.0 | (0.43) | 7.57 | 4.94 | 6.24 | 7.31 | 6.43 | 4.94 |
| control | | | | | | | | | | | | |
| Aerobic | 5.0 | (0.52) | 36.8 | (4.3) | 21.4 | (0.36) | 7.57 | 4.11 | 6.24 | 4.25 | 6.43 | 4.44 |

| control^{d} | | | | | | | | | | |
|---|---|---|---|---|---|---|---|---|---|---|
| Natural | NA | NA | 8.4 | (1.6) | NA | NA | NA | NA | ND | 4.76 |
| ensiling, | | | | | | | | | | |

100 mL

^{a}Represents the average of triplicate experiments, standard deviation of the mean is listed in parenthesis.
^{b}Average of two replicates, third replicate was judged a statistical outlier.
^{c}Average of two replicates.
^{d}Anaerobic and aerobic control experiments were initiated using the same biomass, as a result initial pH measurements are identical.
NA refers to measurements that are not applicable to a particular experiment. ND refers to a measurement that was not determined.

Under aerobic conditions, both the microalgae biomass blends and the corn stover alone had the highest dry matter loss (21.4% and 5.0%) compared to the same materials stored anaerobically (7.0% and 3.4%, respectively), indicating anaerobic storage is a better approach for preservation for the microalgae biomass. The anaerobic control of the microalgae/corn stover blend had a surprisingly low dry matter loss of 7.0% (compared to 43.9% for algae alone stored anaerobically), likely caused by microbial conversion of corn stover soluble sugars to organic acids, dropping the pH from 6.43 to 4.94 to stabilize the biomass. The acidified microalgae/corn stover blends treated with organic acids and sulfuric acids all exhibited dry matter losses lower than 2%. These results further confirm that anaerobic, low pH storage is a promising approach to stabilize microalgae biomass.

Although dry matter loss for corn stover controls, anaerobic- and aerobically stored, were generally low (3.4% and 5.0%, respectively), due to their inherent recalcitrant characteristics, acidification resulted in either similar or reduced dry matter loss (e.g., sulfuric acid pH˜4 and 1% acetic acid, 3% lactic acid). Increases of pH in sulfuric acid and acetic/lactic acid treated microalgae biomass blends and corn stover samples were observed after 30 days storage. However, there were no pH changes in the lactic acid treated microalgae biomass blends and corn stover. For all the acidified, stored microalgae samples, there were obvious pH increases, suggesting different microbial community and degradation activity from the microalgae biomass blends and corn stover alone samples. In general, under anaerobic conditions, when the pH drops and reaches about 3 or 4, the metabolic activity e.g., microbial activity, is highly inhibited.

The proof-of-principle studies demonstrated the effectiveness of low pH and anaerobic conditions to stabilize microalgae biomass blends of microalgae and corn stover. The anaerobic microalgae/corn stover blend achieved surprisingly low dry matter loss when compared with the microalgae only anaerobic control, indicating the potential for microalgae/corn stover blends to naturally ensile.

To be economically viable at commercial scale, microalgae/corn stover blends will need to naturally ensile, producing organic acids needed for preservation in situ. A natural ensiling experiment (6× initial studies) was conducted to evaluate the storage performance of an microalgae/corn stover blend (20% microalgae, db) inoculated with Lactobacillus acidophilus at a rate consistent with forage ensiling to encourage growth of lactic acid bacteria and ensiling. By the end of the 35 day storage period the microalgae/corn stover biomass had lost 8.4% of its initial dry weight and the pH had been reduced below 4.76 (see Table 1), an outcome that is similar to the proof-of-principle anaerobic control. The dry matter loss and final pH were higher for the naturally ensiled sample than observed for the acidified microalgae biomass blends, indicating that more optimization needs to occur to promote greater production of endogenous organic acids. However, losses were within the range of the proof-of-principle anaerobic control and were significantly reduced compared to the aerobic control, indicating that natural ensiling of microalgae and corn stover blends is possible, yet more optimization needs to occur to produce sufficient levels of endogenous acid for preservation.

### Example 5

Analysis of Fermentation Gases

The composition of fermentation gases produced during the storage experiments was determined by gas chromatography using a gas chromatograph (MicroGC 3000, Agilent, Santa Clara, Calif.) equipped with two Molecular Sieve 5 Å columns (10 m length×320 μm ID×12 μm film), one to quantify hydrogen and oxygen (argon carrier gas, 25 psi) and the other to measure methane and carbon monoxide (helium carrier gas, 25 psi) and a Plot U column (6 m length×320 μm ID×30 μm film) (helium carrier gas, 25 psi) to facilitate carbon dioxide measurement. Each column was connected to a thermal conductivity detector, which was calibrated using mixtures of relevant gases. During analysis, the temperature of the column and injector were both held at 60° C. For analysis, a sample of biogas was removed periodically from each serum vial using a gas-tight syringe.

Organic acids present in both fresh and stored biomass samples were extracted using a 1:10 ratio of wet biomass (1 g) to deionized water. The biomass samples were vortexed and equilibrated overnight, and then an aliquot was removed and filtered through a 0.2 μm syringe filter and acidified with 0.1 volume of 4 N H₂SO₄. Each extract was analyzed in duplicate using high-performance liquid chromatography (HPLC) with a refractive index detector (1200 series, Agilent, Santa Clara, Calif.). Individual organic acids were separated using an AMINEX® HPX 87H ion exclusion column (P/N 125-0140, Bio-Rad, Hercules, Calif.).

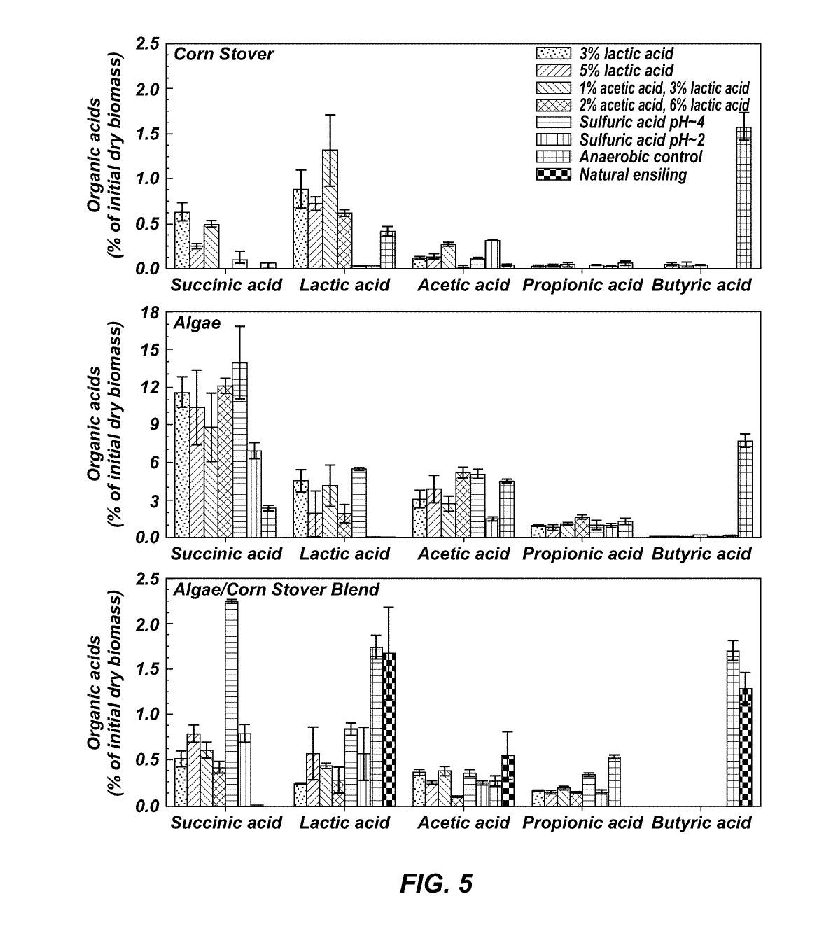

During anaerobic wet storage, fermentative bacteria convert the carbohydrates, proteins, and lipids into volatile fatty acids, such as acetate, propionate, lactate, and butyrate, which facilitate the preservation of the biomass. Lower pH (˜4) serves to inhibit the growth of microorganisms responsible for excessive degradation, including yeast, mold, and bacteria (e.g., Clostridium sp.) Additional acid production was not expected as none of the treatments were inoculated with lactic acid producing bacteria (e.g., Lactobacillus). After 30 days of storage, each biomass sample had more organic acids than initially measured, as shown in FIG. 5. Lactic acid was the primary organic acid produced in the acidified corn stover samples, while succinic acid and acetic acid were produced to a much lesser extent. In the anaerobic corn stover control, butyric acid was the primary fermentation product. This outcome was also observed for the anaerobic control for the microalgae/corn stover blends and the natural ensiling experiment (inoculating the microalgae/corn stover blends with LAB). Butyric acid is an indicator of Clostridia fermentation and is commonly associated with poorly ensiled material. Clostridium sp. consume either soluble sugars or lactic acid to produce butyric acid, hydrogen gas, and carbon dioxide, which leads to high dry matter loss and increased pH. The end application of most ensiled material is for livestock feed, where high butyric acid content reduces feed quality by lowering palatability and decreasing the nutritive value. Beyond higher dry matter loss, the impact of butyric acid fermentation on fuel yield is uncertain. Of the stored corn stover samples, the anaerobic control had the highest initial and final pH. Although real-time pH and organic acid concentrations were not measured, how quickly the pH of a sample is lowered may have an impact on the production of butyric acid by organisms, such as Clostridium sp.

Microalgae-only samples yielded significantly more organic acids than the microalgae/corn stover blends and neat corn stover samples. Particularly, high concentrations of succinic acid, up to 14% on a dry weight basis, were observed from all the microalgae samples. The highest succinic acid production was from the stored microalgae sample treated with a mixture of 2% acetic and 6% lactic acid, resulting in a yield of 0.34 g/g sugar, 25.6% of theoretical glucose conversion to succinic acid (1.33 g/g glucose), assuming total fermentable sugars in algae is 38% dry weight basis. Considering the high global market demand of succinic acid as a valuable bioproduct and intermediate, the conversion of the microalgae biomass during anaerobic storage deserves further investigation. The microalgae/corn stover blends also experienced increased succinic acid production relative to the stored corn stover. The microalgae/corn stover blends treated with sulfuric acid to achieve a pH of ˜4 had the highest succinic acid yield, >2% of biomass (db).

Gas evolution often accompanies organic acid production and consists mostly of carbon dioxide with some observed hydrogen. The carbon dioxide and hydrogen production calculated on the basis of dry biomass for each material and experimental treatment is listed in Table 2. Consistent with the high level of degradation, the microalgae anaerobic control without pH adjustment had the highest observed carbon dioxide production (49.88 ml carbon dioxide/g dry biomass) over 30 days storage. In contrast, each acidified microalgae sample had little (<5 ml/g dry biomass) carbon dioxide production, the lone exception being the sulfuric acid treated sample (pH 4), where a carbon dioxide yield of 9.43 ml/g dry biomass was observed. The carbon dioxide produced from anaerobic controls of the microalgae/corn stover blends and corn stover alone samples was 8.88 and 2.47 ml/g dry biomass, respectively, much lower than the microalgae stored alone. This further demonstrates the benefit to stabilization that blending the microalgae biomass with terrestrial biomass, such as corn stover biomass, had and the potential of this strategy to preserve microalgal biomass quantity and quality.

TABLE 2
Carbon dioxide and hydrogen production for corn stover biomass alone,
microalgae biomass alone, and microalgae/com stover biomass blends.

| | CO₂ Production | | | | | | | | H₂ Production | | | |
|---|---|---|---|---|---|---|---|---|---|---|---|---|
| | (mL/g biomass) | | | | | | | | (mL/g biomass) | | | |
| | | | | | Algae/ | | | | | | Algae/ | |
| Storage | Corn | | | | Corn | | Corn | | | | Corn | |
| Conditions | stover | | Algae | | stover | | stover | | Algae | | stover | |
| 3% lactic acid | 1.32 | (0.02) | 4.41 | (2.61) | 0.32 | (0.20) | 0 | | 0 | | 0 | |
| 5% lactic acid | 0.16 | (0.03) | 3.38 | (3.23) | 0.15 | (0.01) | 0 | | 0 | | 0 | |
| 1% acetic, | 0.09 | (0.02) | 1.81 | (0.27) | 0.15 | (0.00) | 0 | | 0 | | 0 | |
| 3% lacetic acid | | | | | | | | | | | | |
| 2% acetic, | 0.07 | (0.00) | 1.42 | (0.12) | 0.14 | (0.00) | 0 | | 0 | | 0 | |
| 6% lactic acid | | | | | | | | | | | | |
| Sulfuric acid, | ND | | 9.43 | (5.45) | 0.64 | (0.06) | ND | | 0.21 | (0.25) | 0 | |
| pH~4 | | | | | | | | | | | | |
| Sulfuric acid, | 0.10 | (0.01) | 0.52 | (0.30) | 0.31 | (0.10) | 0 | | 0.01 | (0.01) | 0 | |
| pH~2 | | | | | | | | | | | | |
| Anaerobic control | 2.47 | (0.24) | 49.87 | (2.57) | 8.88 | (2.44) | 1.08 | (0.06) | 5.35 | (0.73) | 1.72 | (0.43) |

| | | | | | | | |
|---|---|---|---|---|---|---|---|
| Natural ensiling, | NA | NA | 8.75 | (10.27) | NA | NA | 0 |
| 100 mL | | | | | | | |

^{a}Cumulative gas production (mL/g biomass) monitored over a 30 day period.
Numbers listed in parenthesis represent standard deviation of the mean.

The composition of gas generated throughout storage primarily included carbon dioxide, although hydrogen was produced in the anaerobic control of each material, reaching 5.4 ml/g biomass in the microalgae only samples and 1.7 ml/g biomass and 1.1 ml/g biomass of the microalgae biomass blend and corn stover only, respectively. No methane production was observed in any of the experiments, indicating low methanogen activity under the acidic, anaerobic environment. Certain species of microalgae, including Scenedesmus obliquus used in the current study, contain genes encoding the protein responsible for hydrogen production (hydrogenase), which is expressed under hypoxic conditions independent of illumination. In addition, microalgal biomass has been reported as an interesting alternative substrate for hydrogen production by Clostridium sp., which could be inadvertently introduced into the storage system from the environment. Clostridium sp. commonly are introduced to herbaceous silage piles through harvesting operations that entrain dirt. Once incorporated into a storage pile, Clostridium sp. can flourish, as they are well known to utilize soluble sugars and structural carbohydrates in the biomass to produce hydrogen, organic acids, and alcohols.

### Example 6

Biomass Compositional Changes as a Result of Storage

Unstored and stored microalgae/corn stover blends and corn stover alone samples were ground with a Retsch ZM 200 Ultra Centrifugal Mill (Retsch, Haan, Germany) to pass through a 0.2-mm screen and homogenized prior to conducting proximate and ultimate analyses. For proximate analysis (i.e., moisture, volatile, ash, and fixed carbon content), a LECO TGA701 Thermogravimetric Analyzer (St. Joseph, Mich.) following ASTM D 5142-09 was used (ASTM International, West Conshohocken, Pa.). Briefly, the biomass samples were heated to and maintained at 107° C. until a constant mass was reached under a 10 L/minute nitrogen flow to measure the moisture content. The temperature was then ramped to 950° C. and held for 7 minutes to determine volatiles. After cooling to 600° C., ash content was determined by switching the gas flow to 3.5 L/minute of oxygen and increasing the temperature to 750° C. until a constant mass was reached. Fixed carbon was determined by the weight loss between the volatile and ash measurements. Ultimate analysis, determining elemental C, H, N, and S concentrations, was performed using a LECO TRUSPEC® CHN with S add-on module (St. Joseph, Mich.) following ASTM D5373-10 [27, 28] and ASTM D4239-10 [27] (ASTM International, West Conshohocken, Pa.), respectively. Oxygen was determined by difference. The biomass samples were run in triplicate for proximate, elemental CHN, and elemental S analyses.

Proximate and ultimate analysis was conducted on each of the treated microalgae/corn stover blends before and after 30 days storage to better understand how storage conditions affect the biomass composition. As shown in Tables 3 and 4, the proximate analysis and ultimate analysis revealed that volatile matter decreased significantly as a result of storage in the lactic acid treated samples, low concentration lactic and acetic acid treated samples, sulfuric acid treated samples, and aerobic control samples. Reduced volatile matter is generally associated with decreased acidity, which will reduce corrosion during in conversion, and with increased energy density. The percentage of fixed and total carbon within the microalgae/corn stover blends was affected in some cases by storage. Fixed carbon increased significantly in all but one of the lactic and acetic/lactic acid treated samples. Aerobic storage resulted in a significant decrease in total carbon content of the micronalgae/corn stover blends, as well as an increase in ash content, consistent with the large dry matter loss observed in this biomass during aerobic storage. Wet anaerobic storage of the microalgae/corn stover blends treated with either lactic acid or a mixture of acetic and lactic acids significantly enriched total carbon. The carbon to oxygen (C:O) ratio and carbon to hydrogen (C:H) ratio either increased (organic acids, sulfuric acid pH 4) or remained unchanged (C:O ratio, sulfuric acid pH 2) as a result of storage when treated with acid, while untreated samples experienced a decrease. Higher C:O and C:H ratios result in higher energy densities and thus higher conversion efficiency.

TABLE 3
Proximate analysis of microalgae/corn stover blends
before storage and after 30 days storage.

| | Proximate Analysis | | | | | | | | | | | |
|---|---|---|---|---|---|---|---|---|---|---|---|---|
| | Volatiles (wt %) | | | | Fixed Carbon (wt %) | | | | Ash (wt %) | | | |
| Treatment | Before^{a} | | After^{b} | | Before | | After | | Before | | After | |
| 3% Lactic acid | 80.42* | (0.55) | 79.20 | (0.26) | 14.02 | (0.54) | 15.21* | (0.25) | 5.06 | (0.02) | 5.07 | (0.02) |
| 5% Lactic acid | 80.75* | (0.58) | 79.47 | (0.45) | 14.02 | (0.60) | 15.21 | (0.44) | 5.23 | (0.02) | 5.33 | (0.01) |
| 1% acetic acid, | 80.72* | (0.50) | 79.74 | (0.09) | 14.58 | (0.48) | 15.65* | (0.07) | 4.70 | (0.02) | 4.61 | (0.03) |
| 3% lactic acid | | | | | | | | | | | | |
| 2% acetic acid, | 80.03 | (0.17) | 79.79 | (0.18) | 15.28 | (0.16) | 15.28* | (0.19) | 4.69 | (0.03) | 4.90 | (0.02) |
| 6% lactic acid | | | | | | | | | | | | |
| Sulfuric acid pH 4 | 80.70* | (0.34) | 79.32 | (0.75) | 13.55 | (0.36) | 14.91 | (0.77) | 5.75 | (0.02) | 5.77 | (0.09) |
| Sulfuric acid pH 2 | 77.87 | (0.39) | 78.83 | (0.68) | 16.18 | (0.35) | 15.84 | (0.68) | 5.94 | (0.08) | 5.83 | (0.08) |
| Aerobic control^{c} | 79.67* | (0.17) | 78.00 | (0.30) | 15.60 | (0.18) | 16.08 | (0.29) | 4.73 | (0.01) | 5.92 | (0.02) |
| Anaerobic control^{c} | 79.67 | (0.17) | 79.89 | (0.37) | 15.60 | (0.18) | 14.94 | (0.39) | 4.73 | (0.01) | 5.17 | (0.02) |

^{a}Proximate and ultimate data collected from pre-storage material.
^{b}Proximate and ultimate analysis conducted on material after 30 days of storage for each storage condition.
^{c}Both the aerobic control and anaerobic control utilized the same material. As a consequence, the post-storage proximate and ultimate analysis for each control are compared to proximate are ultimate analysis of the same pre-storage material.
*Denotes measurements determined to be significant by one way, single factor ANOVA analysis.
Numbers listed in parenthesis represent the standard deviation.

TABLE 4
Ultimate analysis of microalgae/corn stover blends before storage and after 30-day storage.

| | Ultimate Analysis | | | | | | | | | | | |
|---|---|---|---|---|---|---|---|---|---|---|---|---|
| | Carbon | | Hydrogen | | Nitrogen | | Oxygen | | C:O | | C:H | |
| | (wt %) | | (wt %) | | (wt %) | | (wt %) | | Ratio | | Ratio | |
| Treatment | Before | After | Before | After | Before | After | Before | After | Before | After | Before | After |
| 3% Lactic | 47.66 | 48.02* | 5.98 | 5.91 | 1.78 | 1.84* | 39.34 | 38.96 | 1.21 | 1.23 | 7.97 | 8.13 |
| acid | (0.02) | (0.01) | (0.10) | (0.01) | (0.02) | (0.01) | (0.10) | (0.03) | | | | |
| 5% Lactic | 47.45 | 47.83* | 5.98 | 5.92 | 1.75 | 1.85* | 39.42 | 38.88 | 1.20 | 1.23 | 7.93 | 8.08 |
| acid | (0.09) | (0.04) | (0.01) | (0.01) | (0.02) | (0.0) | (0.11) | (0.04) | | | | |
| 1% acetic | 47.60 | 48.19* | 6.02 | 5.91 | 1.74 | 1.78* | 39.76 | 39.32 | 1.20 | 1.23 | 7.91 | 8.15 |
| acid, 3% | (0.08) | (0.05) | (0.01) | (0.03) | (0.01) | (0.01) | (0.08) | (0.08) | | | | |
| lactic acid | | | | | | | | | | | | |
| 2% acetic | 47.56 | 48.07* | 6.00 | 5.95 | 1.64 | 1.83* | 39.96 | 39.04 | 1.19 | 1.23 | 7.93 | 8.08 |
| acid, 6% | (0.02) | (0.04) | (0.02) | (0.01) | (0.01) | (0.01) | (0.19) | (0.02) | | | | |
| lactic acid | | | | | | | | | | | | |
| Sulfuric | 47.29 | 47.51 | 5.96 | 5.86 | 1.72 | 1.83* | 38.72 | 38.45 | 1.22 | 1.24 | 7.93 | 8.11 |
| acid pH 4 | (0.20) | (0.10) | (0.07) | (0.03) | (0.02) | (0.01) | (0.13) | (0..01) | | | | |
| Sulfuric | 46.66 | 46.74 | 5.93 | 5.82 | 1.79 | 1.76 | 38.69 | 38.79 | 1.21 | 1.20 | 7.87 | 8.03 |
| acid pH2 | (0.03) | (0.10) | (0.0) | (0.01) | (0.01) | (0.03) | (0.01) | (0.12) | | | | |
| Aerobic | 47.89* | 47.40 | 5.93 | 5.85 | 1.71 | 2.20* | 38.56 | 38.40 | 1.24 | 1.23 | 8.08 | 8.10 |
| control^{c} | (0.02) | (0.05) | (0.06) | (0.02) | (0.03) | (0.01) | (0.06) | (0.08) | | | | |
| Anaerobic | 47.89 | 47.85 | 5.93 | 5.98 | 1.71 | 1.81* | 38.56 | 39.00 | 1.24 | 1.23 | 8.08 | 8.00 |
| control^{c} | (0.02) | (0.03) | (0.06) | (0.0) | (0.03) | (0.01) | (0.06) | (0.03) | | | | |

^{a}Proximate and ultimate data collected from pre-storage material.
bProximate and ultimate analysis conducted on material after 30 days of storage for each storage condition.
^{c}Both the aerobic control and anaerobic control utilized the same material. As a consequence, the post-storage proximate and ultimate analysis for each control are compared to proximate and ultimate analysis of the same pre-storage material.
*Denotes measurements determined to be significant by one way, single factor ANOVA analysis.
Numbers listed in parenthesis represent the standard deviation.

Nitrogen was significantly enriched as a result of storage with the exception of the sulfuric acid pH 2 sample. The relatively high concentration of nitrogen in the biomass is a concern for thermochemical conversion, where nitrogen reduces the quality of oil produced and necessitates additional processing steps to remove it. No significant difference in sulfur content was observed as a result of storage. The sulfur content was elevated in storage treatments which were treated with sulfuric acid, relative to other treatments and controls. Sulfuric acid treatments may be less desirable for thermochemical approaches to fuel conversion, such as hydrothermal liquefaction, due to potential sulfur emissions but may be beneficial to biochemical conversion strategies that rely on dilute acid pretreatment with sulfuric acid in order to make sugars more accessible for conversion. Storage did not appear to significantly affect the hydrogen or oxygen content.

### Example 7

N₂ or CO₂ Purge Experiments

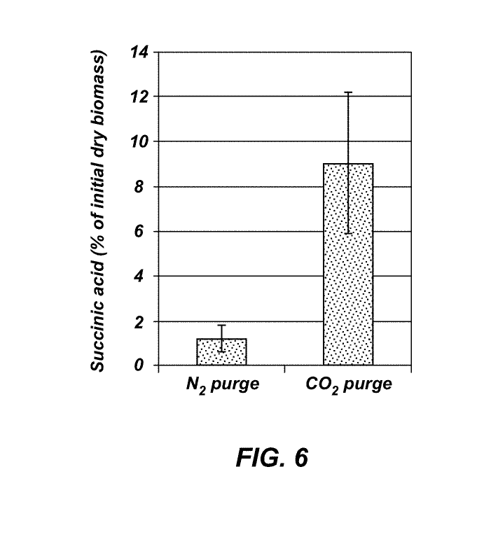

Acidified microalgae biomass samples were purged with nitrogen gas (N₂) or carbon dioxide (CO₂) gas. The microalgae biomass was produced as described above in Example 1 and was acidified with 0.5% by weight sulfuric acid followed by either a single N₂ purge or a single CO₂ purge prior to the 30 day storage period. As shown in FIG. 6, the microalgae biomass sample purged with CO₂ produced significantly more succinic acid than the sample purged with N₂. About 9% succinic acid was produced following the CO₂ purge, compared to about 1% succinic acid following the N₂ purge.

While the disclosure may be susceptible to various modifications and alternative forms, specific embodiments have been shown by way of example in the Examples and drawings and have been described in detail herein. However, it should be understood that the disclosure is not intended to be limited to the particular forms disclosed. Rather, the disclosure is to cover all modifications, equivalents, and alternatives falling within the scope of the disclosure as defined by the following appended claims and their legal equivalents.

What is claimed is:

1. A method of preserving a biomass, the method comprising:
adding an acid solution to a biomass comprising microalgae to form an acidified microalgae biomass composition; and
storing the acidified microalgae biomass composition under anaerobic conditions without inoculating the acidified microalgae biomass composition with bacteria.

2. The method of claim 1, wherein adding an acid solution to a biomass comprising microalgae comprises adding the acid solution to the biomass comprising microalgae biomass and herbaceous biomass.

3. The method of claim 1, wherein adding an acid solution to a biomass comprising microalgae comprises adding the acid solution to the biomass comprising a pH of greater than about 6.0.

4. The method of claim 1, wherein adding an acid solution to a biomass comprising microalgae comprises adding lactic acid, acetic acid, combinations thereof, or sulfuric acid to the biomass.

5. The method of claim 1, further comprising exposing the acidified microalgae biomass composition to carbon dioxide, nitrogen, or a combination thereof.

6. A method of preserving a biomass, the method comprising:
adding an acid solution to a biomass comprising microalgae to form an acidified microalgae biomass composition;
storing the acidified microalgae biomass composition under anaerobic conditions; and
exposing the acidified microalgae biomass composition to carbon dioxide, nitrogen, or a combination thereof to produce a coproduct comprising succinic acid.

7. The method of claim 6, wherein adding an acid solution to a biomass comprising microalgae comprises adding the acid solution to the biomass comprising at least one microalgal strain.

8. The method of claim 6, wherein adding an acid solution to a biomass comprising microalgae comprises adding the acid solution to the biomass comprising Scenedesmus.

9. The method of claim 6, wherein adding an acid solution to a biomass comprising microalgae comprises adding the acid solution to the biomass comprising Scenedesmus and corn stover.

10. The method of claim 6, wherein adding an acid solution to a biomass comprises forming the acidified microalgae biomass composition comprising a pH of from about 2.0 to about 5.0.

11. The method of claim 6, wherein adding an acid solution to a biomass comprises forming the acidified microalgae biomass composition comprising a pH of from about 2.5 to about 4.5.

12. The method of claim 6, wherein adding an acid solution to a biomass comprises adding an organic acid solution comprising at least one organic acid to the biomass.

13. The method of claim 6, wherein adding an acid solution to a biomass comprises adding an inorganic acid solution comprising at least one inorganic acid to the biomass.

14. The method of claim 6, wherein storing the acidified microalgae biomass composition under anaerobic conditions comprises storing the acidified microalgae biomass composition under the anaerobic conditions for at least about 30 days.

15. The method of claim 6, wherein adding an acid solution to a biomass comprising microalgae comprises adding the acid solution to the biomass without inoculating the biomass with a bacteria.

16. The method of claim 6, wherein producing a coproduct comprising succinic acid comprises producing the succinic acid in-situ by the biomass comprising microalgae.

17. The method of claim 6, wherein producing a coproduct comprising succinic acid comprises producing the succinic acid while exhibiting a dry matter loss of the biomass of less than about 5% dry basis.

18. The method of claim 6, further comprising recovering the succinic acid.

19. A method of preserving a biomass, the method comprising:
adding an acid solution to a biomass comprising microalgae to form an acidified microalgae biomass composition; and
storing the acidified microalgae biomass composition under anaerobic conditions and in the presence of carbon dioxide.

20. The method of claim 19, wherein storing the acidified microalgae biomass composition under anaerobic conditions and in the presence of carbon dioxide comprises storing the acidified microalgae biomass composition in an environment consisting of carbon dioxide.

21. The method of claim 19, wherein storing the acidified microalgae biomass composition under anaerobic conditions and in the presence of carbon dioxide comprises storing the acidified microalgae biomass composition in a vessel comprising a headspace and the carbon dioxide.

22. A preserved biomass, comprising:
a biomass comprising microalgae and succinic acid, the succinic acid comprising up to about 14% of the biomass on a dry weight basis.

23. The preserved biomass of claim 22, wherein the succinic acid comprises from about 6% of the biomass on a dry weight basis to about 14% of the biomass on a dry weight basis.

24. The preserved biomass of claim 22, wherein the preserved biomass comprises a dry matter loss of less than about 5% dry basis.

25. The preserved biomass of claim 22, further comprising a herbaceous biomass.

26. The preserved biomass of claim 25, wherein the microalgae comprises Scenedesmus and the herbaceous biomass comprises corn stover.
